(19) United States
(12) Patent Application Publication
(10) Pub. No.: US 2017/0143597 A1
(43) Pub. Date: May 25, 2017

(54) COMPOSITIONS FOR COSMETIC RAW MATERIAL AND METHODS FOR MAKING THE SAME

(71) Applicant: KPT LTD., Cheongju-Si, Chungcheongbuk-do (KR)

(72) Inventors: Yanfu JIANG, Yongin-si, Gyeonggi-do (KR); Ik Joo LEE, Ansan-si, Gyeonggi-do (KR); Woon Jang LEE, Cheongju-si, Chungcheongbuk-do (KR); Byung-ho PARK, Cheongju-si, Chungcheongbuk-do (KR); Jae Uk LEE, Daejeon (KR)

(73) Assignee: KPT LTD., Cheongju-Si, Chungcheongbuk-do (KR)

(21) Appl. No.: 15/108,944

(22) PCT Filed: Apr. 30, 2015
(86) PCT No.: PCT/IB2015/053182

§ 371 Date: Jun. 29, 2016

(51) Int. Cl. A61K 8/11; A61K 8/34; A61K 8/73; A61K 8/25; B01J 13/04; A61K 8/55; A61K 8/81; A61Q 1/02; A61Q 19/00; A61K 8/02; A61K 8/29

(52) U.S. Cl. CPC A61K 8/11; A61K 8/025; A61K 8/0279; A61K 8/345; A61K 8/731; A61K 8/25; A61K 8/29; A61K 8/553; A61K 8/732; A61K 8/8147; A61Q 1/02; A61Q 19/00; B01J 13/04; A61K 2800/41; A61K 2800/621; A61K 2800/622; A61K 2800/624; A61K 2800/63; A61K 2800/652; A61K 2800/654

(57) ABSTRACT

The instant invention relates to a composition for cosmetic raw material containing microcapsule containing at least one encapsulated material comprising at least one core and at least one layered coating surrounding the core, and the encapsulated material being at least one particle having a high wet point and being optionally porous, and being only released from the microcapsule when the composition is applied onto a keratin material, such as keratin fibers or skin. The invention further relates to a method for preparing the composition for cosmetic raw material containing microcapsule containing at least one encapsulated material comprising at least one core and at least one layered coating surrounding the core, and the encapsulated material being at least one particle having a high wet point and being optionally porous, and being only released from the microcapsule when the composition is applied onto a keratin material, such as keratin fibers or skin.

### FIELD OF THE INVENTION

The present invention relates to compositions for cosmetic raw material comprising microcapsules containing at least one particle having a high wet point and methods for making the same.

### BACKGROUND OF THE INVENTION

There is a growing interest in imparting care properties in cosmetic products especially in make-up compositions. These care properties are often associated with a smooth, creamy, rich appearance of the compositions.

Nevertheless, in particular, the introduction of some ingredients in cosmetic compositions may be detrimental towards the texture of the composition.

In particular, the introduction of some ingredients in cosmetic compositions may be detrimental towards the stability and especially the rheology of the composition.

Finally, the introduction of some ingredients in cosmetic compositions may be detrimental towards the general appearance and comfort of use of the composition, in particular for skin-care products for which it is generally sought some codes which are an aesthetical purity of the composition associated with a good texture when the composition is picked up and applied onto the skin.

As representative of this kind of ingredients may be in particular mentioned some particle having a high wet point and being optionally porous which undesirably may act as rheology modifiers when used in too important amounts.

Indeed they can absorb a significant part of the composition in which they are introduced, this absorption leading to a thickening of the composition which may be undesirable.

Accordingly, there is a need for compositions containing particles having a high wet point and being optionally porous, but which rheological properties are not modified by the presence of such particles.

There is also a need for compositions allowing to provide to the user, the benefit of high amounts particle having a high wet point but in contrast being free from the undesirable effect with respect to their rheologic properties especially not presenting a gritty feeling.

Surprisingly and advantageously, the compositions according to the invention meet these needs.

### SUMMARY OF INVENTION

According to one of its aspects, the invention is directed to a microcapsule composition containing at least one core and at least one layered coating surrounding the core, and the encapsulated material(s) being at least one particle having a high wet point and being optionally porous.

According to another embodiment, the invention is directed to a microcapsule containing at least one core and at least one layered coating surrounding the core, and the encapsulated material(s) being at least one particle having a high wet point and being optionally porous.

Another aspect of the present invention is a method of preparing the microcapsules, microparticles or encapsulated particle. For example, the method includes:

preparing an aqueous solution containing water, a lower alcohol such as ethanol, and a hydrophilic gelling agent which is soluble in water and the alcohol,

dispersing an aerogel and optionally a pigment in the aqueous solution; and

coating a core with the aqueous solution.

The solution for coating the core may not contain water. For example, the solution can contain the lower alcohol and the hydrophilic gelling agent without water. Preferably, the solution for coating the core does not contain a hydrophobic solvent.

### BRIEF DESCRIPTION OF THE DRAWINGS

FIG. 1 is a schematic diagram illustrating a typical structure of a microcapsule of the present invention, wherein A represents a core and B, and C, being different layers concentrically surrounding the core.

FIG. 1 typically represents the microcapsule of example 12 wherein A represents the core comprising lecithin, mannitol, a corn starch binder and reflective particle(s), B represents the inner layer comprising lecithin, mannitol, a corn starch binder and reflective particle(s) and C represents the outer layer comprising lecithin and a corn starch binder.

### DETAILED DESCRIPTION OF THE INVENTION

According to one of its aspects, the invention is directed to a microcapsule composition containing at least one core and at least one layered coating surrounding the core, and the encapsulated material(s) being at least one particle having a high wet point and being optionally porous.

According to another embodiment, the invention is directed to a microcapsule containing at least one core and at least one layered coating surrounding the core, and the encapsulated material(s) being at least one particle having a high wet point and being optionally porous.

Another aspect of the present invention is a method of preparing the microcapsules, microparticles or encapsulated particle. For example, the method includes:

preparing an aqueous solution containing water, a lower alcohol such as ethanol, and a hydrophilic gelling agent which is soluble in water and the alcohol,

dispersing an aerogel and optionally a pigment in the aqueous solution; and

coating a core with the aqueous solution.

The solution for coating the core may not contain water. For example, the solution can contain the lower alcohol and the hydrophilic gelling agent without water. Preferably, the solution for coating the core does not contain a hydrophobic solvent.

The microparticle is also expressed as microcapsule or encapsulated particle.

The microcapsules according to the invention are particularly interesting for the following reasons.

The encapsulated particles having a high wet point and being optionally porous, are kept in the microcapsules during the storage of the composition and only released upon application of the composition on the keratin material.

By this way, the microcapsules according to the invention allow to permanently retain the particles having a high wet point and being optionally porous, in the microcapsule during the storage of the composition, and thus to efficiently prevent any undesirable modification of the stability of the composition and to keep a same long-term visual effect to the composition.

The microcapsules according to the invention are also advantageously stable with a large panel of solvent/ingredient associated.

They are also stable into the compositions according to the present invention, preferably at high temperatures, for instance greater than or equal to 40° C., for example for one month, better two months and still better three months in an oven at 45° C. or for 15 days in an oven at 60° C.

In a preferred embodiment, the microcapsules according to the present invention present an appropriate softening kinetics.

That is preferably, at least three hours after being in contact with the other compounds of the formula, the hardness of the microcapsules is advantageously from 5 to 50 grams, more preferably from 6 to 20 grams and still more preferably from 7 to 10 grams. Such hardness is in conformity with an industrial process for preparing the cosmetic compositions including such microcapsules.

Such values of softening kinetics and hardness allow to provide not only aesthetic microcapsules but also overall aesthetic compositions.

The microcapsules are not visible inside the bulk of the composition depending on the desired appearance.

Advantageously, they have the ability of swelling or softening in contact of a liquid medium such as water and optionally at least one compound chosen from polyols, glycols and C₂-C₈ monoalcohols, and mixtures thereof or alternatively in a liquid fatty phase preferably an oily phase. By this way, they are advantageously deformable when applied on a keratin material and consequently provide a soft feeling to the user.

Furthermore, their size contributes to not create any discomfort or unfavorable, grainy feeling when applied. In particular, they are soft enough to rupture upon very slight rubbing or pressing on the skin in order to release their content.

They disintegrate rapidly immediately when applied, with a liquid feeling on the skin and leading to compositions devoid of any granular aspect.

However, they are durable enough to avoid destruction of the coating during manufacture, even during an industrial process, and storage of corresponding composition. Thus, they exhibit a hardness sufficient to be compounded in an industrial process without alteration. Advantageously the hardness of the microcapsules does not significantly decrease during the preparation process. Thus, they allow the use of regular equipment for the preparation of the compositions of the invention.

Accordingly, the microcapsules of the present invention are particularly interesting since they increase the stability of the particles having a high wet point and being optionally porous, against degradation, and prevent undesirable release of the encapsulated actives into the composition during the manufacturing process and prolonged storage.

Another aspect of the present invention is a method of preparing the microcapsules, microparticles or encapsulated particle. For example, the method includes:

preparing an aqueous solution containing water, a lower alcohol such as ethanol, and a hydrophilic gelling agent which is soluble in water and the alcohol,

dispersing an aerogel and optionally a pigment in the aqueous solution; and

coating a core with the aqueous solution.

The solution for coating the core may not contain water. For example, the solution can contain the lower alcohol and the hydrophilic gelling agent without water. Preferably, the solution for coating the core does not contain a hydrophobic solvent.

A composition or a microcapsule according to one aspect of the invention may comprise from 0.1% to 20% by weight and preferably from 0.5% to 15% by weight of microcapsules relative to the total weight of the composition.

In particular for a skin care composition according to the invention, the amount of microcapsules will range from 0.1% to 5%, preferably from 0.2% to 3% by weight relative to the total weight of composition.

In particular for a make-up composition according to the invention, the amount of microcapsules will range from 0.5% to 20%, preferably from 1% to 15%, more preferably from 2% to 10% by weight relative to the total weight of composition.

Advantageously, a composition of the invention may comprise two or more microcapsules of the invention different from each other.

According to a preferred embodiment, the particle(s) having a high wet point is (are) porous.

According to a preferred embodiment, the particle(s) having a high wet point at least for oil(s), and preferably for oil(s) and for water. The methods for valuating this wet point is further detailed later in the description.

According to a preferred embodiment, the particle(s) having a high wet point is (are) porous and have a high wet point at least for oil(s), and preferably for oil(s) and for water.

According to a first embodiment, the encapsulated particle(s) having a high wet point and being optionally porous, is/are present in the core of the microcapsules. Particularly, the encapsulated particle having a high wet point, which is/are optionally porous, is/are only present in the core of the microcapsules.

In one specific sub-embodiment, the core of the microparticles includes the particle(s) having a high wet point and being optionally porous, and at least one binder.

In another specific sub-embodiment, the particles having a high wet point and being optionally porous, is/are present in the core as a lipid or aqueous dispersion.

According to a second embodiment, at least one inner layer surrounding the core includes the encapsulated particle(s) having a high wet point and being optionally porous.

Inner layer means that this layer is obligatory surrounded by another, inner or outer, layer. Further the layered coating advantageously comprises at least one inner layer and one outer layer.

Particularly, the encapsulated particle having a high wet point, being optionally porous, is/are only present in at least one inner layer of the microcapsules.

The term “encapsulated” means that the particle having a high wet point, optionally porous, is always entrapped inside the microcapsules according to the invention.

In other words, the outer layer of the microcapsules encapsulating the particle having a high wet point and being optionally porous, is always free from any particle having a high wet point and being optionally porous. Advantageously, the outer layer is free from particle having a high wet point and being optionally porous, and preferably comprises at least one hydrophilic polymer and optionally a binder.

According to a third embodiment, the encapsulated particle having a high wet point and being optionally porous, is present in the core of the microcapsules and in at least on inner layer.

Chemical Nature of Microcapsules

According to a preferred embodiment, the core is an organic core.

The core of the microparticles may consist in at least one or several particle(s) having a high wet point and being optionally porous. If the core is not totally made of particles having a high wet point and being optionally porous, it comprises additional organic material(s).

Advantageously the core represents from 1% to 50% by weight, preferably 5 to 30% by weight, and in particular from 10 to 20% by weight relative to the total weight of the microcapsule.

Preferably the microcapsules have a double layer surrounded the core.

Preferably, the microcapsules contain at least one organic layer, preferably one inner organic layer.

According to a preferred embodiment, the microcapsules contain at least one layer, preferably at least one inner layer, comprising at least one binder.

According to another embodiment the outer layer comprises a binder.

Advantageously, the microcapsules have a size of from 50 μm to 800 μm, in particular from 60 μm to 600 μm, and in particular from 80 μm to 500 μm, and in particular from 100 μm to 400 μm.

Preferably the microcapsule comprises at least 5%, preferably at least 10%, more preferably at least 30%, better at least 40%, even better at least 50%, advantageously at least 60% and in particular between 30 and 80% preferably between 40 and 75% by weight of particle having a high wet point and being optionally porous, relative to the weight of the microcapsule.

According to a preferred embodiment, the microcapsules comprise:
a core comprising at least one particle having a high wet point and being optionally porous, and optionally at least one additional organic material,
at least one layered coating surrounding the core, the layered coating comprising a binder selected from at least one polymer, at least one lipid-based material, and their mixture, preferably their mixture and optionally at least one particle having a high wet point and being optionally porous, which may be the same or different from the particle having a high wet point contained in the core,
an outer layer comprising a hydrophilic polymer.

According to another preferred embodiment, the microcapsules comprise
a core comprising at least one organic material,
at least one layered coating surrounding the core, the layered coating comprising a binder selected from at least one polymer, at least one lipid-based material, and their mixture, preferably their mixture and at least one particle having a high wet point and being optionally porous,
an outer layer comprising a hydrophilic polymer.

Preferably, the core comprises at least one monosaccharide or its derivatives as the organic material, in particular a monosaccharide-polyol advantageously selected from mannitol, erythritol, xylitol, sorbitol and mixtures thereof, preferably mannitol.

Preferably, the layered coating surrounding the core comprises at least one hydrophilic polymer(s) selected from the group consisting of:
acrylic or methacrylic acid homopolymers or copolymers or salts and esters thereof
copolymers of acrylic acid and of acrylamide and its salts and esters thereof
polyhydroxycarboxylic acids and its salts and esters thereof
polyacrylic acid/alkyl acrylate copolymers, preferably modified or unmodified carboxyvinyl polymers;
AMPS;
AMPS/acrylamide copolymers;
polyoxyethylenated AMPS/alkyl methacrylate copolymers;
anionic, cationic, amphoteric or nonionic chitin or chitosan polymers;
cellulose polymers and derivatives;
starch polymers and derivatives, eventually modified;
vinyl polymers and derivatives;
polymers of natural origins and derivatives thereof
alginates and carrageenans;
glycoaminoglycans, hyaluronic acid and derivatives thereof
mucopolysaccharides such as hyaluronic acid and chondroitin sulfates;

and the mixtures thereof.

Advantageously the layered coating comprises at least hydrophilic polymer(s) selected from the group consisting of polysaccharides and derivatives, acrylic or methacrylic acid homopolymers or copolymers or salts and esters thereof, and their mixture; the polysaccharides and derivatives are preferably selected from chitosan polymers, chitin polymers, cellulose polymers, starch polymers, galactomannans, alginates, carrageenans, mucopolysaccharides, and their derivatives, and the mixture thereof, more preferably starch polymers and derivatives, cellulose polymers and derivatives, and their mixture.

Particularly the hydrophilic polymer(s) is selected from the polysaccharides and derivatives including one type of ose or several type of ose(s), preferably several type of ose(s) including at least D-glucose units.

Particularly the hydrophilic polymer is selected from starch or derivatives, celluloses or derivatives, preferably starch or derivatives.

Preferably, the core comprises at least one monosaccharide polyol, preferably selected from mannitol, erythritol, xylitol, sorbitol, and the layered coating comprises at least one polysaccharides (or its derivatives) including as oses at least D-Glucose unit(s), preferably selected from starch or derivatives, celluloses or derivatives, preferably starch or derivatives.

Preferably the outer layer of microcapsule is free from particle having a high wet point and being optionally porous and preferably comprises at least one hydrophilic polymer and optionally a binder.

Preferably the outer layer comprising at least one hydrophilic polymer defined in the above list. Preferably this hydrophilic polymer is at least one wall-forming polymer preferably selected from polysaccharides such as cellulose derivatives, in particular cellulose ether and cellulose ester, from (poly)(alkyl)(meth)acrylic acid and derivatives, notably (poly)(alkyl)(meth)acrylate and derivatives, and preferably from alkylacrylic/alkylmethacrylic acid copolymers and their derivatives.

Preferably, the microcapsules include at least one lipid based material, preferably with amphiphilic properties such as lecithins and in particular hydrogenated lecithin.

For the purposes of the present invention, the term “keratin material” is intended to cover the skin, mucous membranes such as the lips, the nails and the eyelashes. The skin and the lips, in particular facial skin, are most particularly considered according to the invention.

I. Microcapsules

The term “microcapsule”, as used herein, refers to a spherical microcapsule containing at least one layered coating and surrounding a core chemically different from the coating. Microcapsules are distinct from microspheres, which consist of spherical homogeneous matrix.

According to an embodiment, the “at least one layered coating” is a multi-layered coating preferably an organic multi-layered coating.

The term “multi-layer microcapsule” refers to a microcapsule consisting of a core surrounded by a coating based on one or more inner layer(s) and one outer layer. The one or more inner layer(s) forming the multi-layer coating of the multi-layer microcapsule and the single outer layer of the microcapsule may be formed of the same or different wall-forming organic compound(s).

The microcapsule according to the invention comprises a core also called “inner core” surrounded by a coating based on one or more layer(s). In a preferred embodiment, the microcapsule is a ‘multi-layers’ microcapsule, comprising at least one inner layer and one outer layer. The one or more inner layer(s) forming the multi-layer coating of the multi-layer microcapsule and the single outer layer of the microcapsule may be formed of the same or different wall-forming organic compound(s).

In a particular embodiment the inner layer and the outer layer are formed of the same wall forming organic compounds, the core is then surrounded by a one layer coating.

The term “wall-forming organic compound” refers to an organic compound or a combination of two or more different organic compounds as defined herein, which form a component of the layer(s) of the microcapsules. In a preferred embodiment, the ‘wall-forming organic compound’ comprises at least one polymer.

Generally, average particle sizes of up to about 800 μm in diameter of microcapsules are used according to the invention. Preferably the average particle size is less than about 400 μm in diameter of the microcapsules for skin care applications. Advantageously the average particle size is in the range of about 10 μm to 350 μm in diameter. Preferably, the average particle size will be from 50 μm to 800 μm, in particular from 60 μm to 600 μm, and in particular from 80 μm to 500 μm, and in particular from 100 μm to 400 μm in diameter.

In particular, the average particle size may be from 50 to 1,000 Mesh (around 400 μm to 10 μm), in particular from 60 to 200 Mesh (around 250 μm to 75 μm) as measured by the sieving test method or observed by microscope.

Ia) Core

The core is made of particle(s) having a high wet point and being optionally porous, and/or of at least an organic material. The size of the core preferably ranges from 500 nm to 150 μm in diameter.

Preferably the core is in a solid and/or crystal form at room temperature.

In a particular embodiment, the organic material is selected from organic materials having high water dissolvability. Preferably, the core is water-soluble or water-dispersible.

In a particular embodiment, the core is based on only one compound, preferably one organic compound.

This compound may be a particle having a high wet point and being optionally porous.

This compound may be a natural compound.

According to a preferred embodiment, the core is sugar-alcohol, preferably a monosaccharide-polyol advantageously selected from mannitol, erythritol, xylitol and sorbitol.

In a particular embodiment, the core is made of mannitol and more preferably exclusively made of mannitol.

According to an alternative embodiment, the core contains at least mannitol and at least one additional ingredient being preferably a polymer selected from hydrophilic polymers. In particular, such a core may comprise mannitol and hydrophilic polymers chosen among cellulose polymers, starch polymers and their mixture, preferably their mixture.

In a preferred embodiment, the cellulose polymer is a carboxymethylcellulose and the starch polymer is a non-modified natural starch, for example corn starch.

The core may be constituted by a seed (or crystal) of one of the previous materials.

The core is preferably contained in an amount of from 1% to 50% by weight, preferably 4 to 40% by weight, in particular 5 to 30% by weight, and in particular from 10 to 20% by weight with respect to the total weight of the microcapsule.

The mannitol is preferably contained in an amount of from 2% to 100% by weight, preferably 5 to 100% by weight, and in particular 100% by weight with respect to the total weight of the core.

The mannitol is preferably contained in an amount of from 1% to 50% by weight, preferably 4% to 40% by weight, in particular 5% to 30% by weight, and in particular from 10% to 20% by weight with respect to the total weight of the microcapsule.

Ib) External Layer(s) or Coating

As disclosed previously, the core is advantageously surrounded with a coating, or external layer(s) preferably comprising at least one inner layer and one outer layer. In this latter case, these layers preferably extend concentrically in respect with the core. The layer(s) is/are preferably organic, i.e. contain(s) at least one organic compound as wall-forming material. Preferably, the inner and/or outer layer(s) include(s) at least one polymer, and in particular a hydrophilic polymer.

Polymer(s)

The composition according to the invention comprises one or more polymer(s). In a particular embodiment, the polymer(s) is/are hydrophilic polymer(s).

Such hydrophilic polymer(s) is/are soluble or dispersible in water or in alcohol compounds, in particular chosen from lower alcohols, glycols, polyols.

For the purposes of the present patent application, the term “hydrophilic polymer” means a (co)polymer that is capable of forming hydrogen bond(s) with water or alcohol compounds, in particular chosen from lower alcohols, glycols, polyols. In particular, polymers are concerned which are capable of forming O—H, N—H and S—H bonds.

According to a particular embodiment of the invention, the hydrophilic polymer may swell or soften in contact with water or alcohol compounds, in particular chosen from lower alcohols, glycols, polyols.

The hydrophilic polymer(s) may be chosen from the following polymer(s):
acrylic or methacrylic acid homopolymers or copolymers or salts and esters thereof and in particular the products sold under the names Versicol F or Versicol K by the company Allied Colloid, Ultrahold 8 by the company Ciba-Geigy, and polyacrylic acids of Synthalen K type, and salts, especially sodium salts, of polyacrylic acids (corresponding to the INCI name sodium acrylate copolymer) and more particularly a crosslinked sodium polyacrylate (corresponding to the INCI name sodium acrylate copolymer (and) caprylic/capric triglycerides) sold under the name Luvigel EM by the company;
copolymers of acrylic acid and of acrylamide sold in the form of the sodium salt thereof under the names Reten by the company Hercules, the sodium polymethacrylate sold under the name Darvan No. 7 by the company Vanderbilt, and the sodium salts of polyhydroxycarboxylic acids sold under the name Hydagen F by the company Henkel;
polyacrylic acid/alkyl acrylate copolymers, preferably modified or unmodified carboxyvinyl polymers; the copolymers most particularly preferred according to the present invention are acrylate/C₁₀-C₃₀-alkylacrylate copolymers (INCI name: Acrylates/C₁₀₋₃₀ Alkyl acrylate Crosspolymer) such as the products sold by the company Lubrizol under the trade names Pemulen TR1, Pemulen TR2, Carbopol 1382 and Carbopol ETD 2020, and even more preferentially Pemulen TR-2;
alkylacrylic/alkylmethacrylic acid copolymers and their derivatives notably their salts and their esters, such as the copolymer of ethyl acrylate, methyl methacrylate and low content of methacrylic acid ester with quaternary ammonium groups provided under the tradename of EUDRAGIT RSPO from Evonik Degussa;
AMPS (polyacrylamidomethylpropanesulfonic acid partially neutralized with aqueous ammonia and highly crosslinked) sold by the company Clariant;
AMPS/acrylamide copolymers such as the products Sepigel or Simulgel sold by the company SEPPIC, especially a copolymer of INCI name Polyacrylamide (and) C13-14 Isoparaffin (and) Laureth-7;
polyoxyethylenated AMPS/alkyl methacrylate copolymers (crosslinked or non-crosslinked) of the type such as Aristoflex HMS sold by the company Clariant;
polysaccharides and derivatives, such as:
anionic, cationic, amphoteric or nonionic chitin or chitosan polymers;
cellulose polymers and derivatives, preferably other than alkylcellulose, chosen from hydroxyethylcellulose, hydroxypropylcellulose, hydroxymethylcellulose, hydroxypropylmethylcellulose, ethylhydroxyethylcellulose and carboxymethylcellulose, and also quaternized cellulose derivatives; in a preferred embodiment, the cellulose polymers is a carboxymethylcellulose;
starch polymers and derivatives, eventually modified; in a preferred embodiment, the starch polymer is a natural starch;
optionally modified polymers of natural origin, such as galactomannans and derivatives thereof, such as konjac gum, gellan gum, locust bean gum, fenugreek gum, karaya gum, gum tragacanth, gum arabic, acacia gum, guar gum, hydroxypropyl guar, hydroxypropyl guar modified with sodium methylcarboxylate groups (Jaguar XC97-1, Rhodia), hydroxypropyltrimethylammonium guar chloride, and xanthan derivatives;
alginates and carrageenans;
glycoaminoglycans, hyaluronic acid and derivatives thereof;
mucopolysaccharides such as hyaluronic acid and chondroitin sulfates, and mixtures thereof;
vinyl polymers, for instance polyvinylpyrrolidones, copolymers of methyl vinyl ether and of malic anhydride, the copolymer of vinyl acetate and of crotonic acid, copolymers of vinylpyrrolidone and of vinyl acetate; copolymers of vinylpyrrolidone and of caprolactam; polyvinyl alcohol;

and the mixtures thereof.

Preferably, the composition according to the invention, and in particular the external layer(s) comprise(s) hydrophilic polymers selected from the group consisting of polysaccharides and derivatives, acrylic or methacrylic acid homopolymers or copolymers or salts and esters thereof, and their mixture.

The polymer(s) is (are) advantageously selected from (poly)(alkyl)(meth)acrylic acid and derivatives, notably (poly)(alkyl)(meth)acrylate and derivatives, preferably from alkylacrylic/alkylmethacrylic acid copolymers and their derivatives, and most preferably is a copolymer of ethyl acrylate, methyl methacrylate and low content of methacrylic acid ester with quaternary ammonium groups provided under the tradename of EUDRAGIT RSPO from Evonik Degussa.

The polysaccharides and derivatives are preferably selected from chitosan polymers, chitin polymers, cellulose polymers, starch polymers, galactomannans, alginates, carrageenans, mucopolysaccharides, and their derivatives, and the mixture thereof.

In a preferred embodiment, the external layer(s) is/are devoid of microcrystalline cellulose.

According to one particularly preferred embodiment, the polysaccharides and their derivatives are preferably selected from the ones including one type of ose or several type of ose(s), preferably several types of oses, in particular at least D-Glucose unit(s) as ose(s), preferably starch polymers, cellulose polymers, and derivatives, and the mixture thereof.

According to a preferred embodiment, the microcapsule contains at least one hydrophilic polymer selected from the group consisting of starch and its derivatives, in particular corn starch, cellulose and its derivatives, homo- and/or co-polymer of methacrylic acid and/or methacrylic acid ester or co-polymer of (alkyl)acrylic acid and/or (alkyl)methacrylic acid and their derivatives, preferably their salts and their ester, and in particular the capsule contains polymethyl methacrylate.

Starch usable according to the present invention is usually issued from vegetable raw materials, such as rice, soybeans, potatoes, or corn. Starch can be unmodified or (by analogy with cellulose) modified starch. In a preferred embodiment, the starch is unmodified.

Preferred homo- and/or co-polymer of methacrylic acid and/or methacrylic acid ester are those wherein the copolymer of methyl methacrylate and ethyl acrylate has a molecular weight from 750 to 850 kDa.

Cellulose derivatives include, for example, alkali celluloses carboxymethyl cellulose (CMC), cellulose esters and ethers, and aminocelluloses. In a particular embodiment, the cellulose is a carboxymethyl cellulose (CMC).

According to a preferred embodiment, the capsule contains at least starch derivative, in particular corn starch, polymethyl methacrylate, co-polymer of (alkyl)acrylic acid and/or (alkyl)methacrylic acid and their derivatives preferably their salts and their ester, and/or cellulose derivative.

Preferably, the microcapsule contains polymer(s) which are not cross-linked.

The polymer(s) may be in one or several layer(s).

In another embodiment, the polymer(s) may be in the core.

The microcapsule may contain polymer(s) in the core and/or in the layer(s).

In a particular embodiment, the polymer(s) is (are) in the core and in the layer(s).

In an embodiment, the core contains at least starch and/or cellulose derivative as polymer(s). When the starch is contained within the core, it represents the main ingredient of such a core, i.e. the weight amount of starch is greater than the respective amount of other compounds of the core.

The polymer may represent from 0.5 to 20% by weight of the microcapsule, in particular from 1 to 10% by weight, preferably from 2 to 8% by weight of the microcapsule.

The different layers forming the coating may be based on identical or different polymers. Advantageously, they will be formed from the same polymer.

The microcapsules advantageously comprises at least:
a core made of at least one particle having a high wet point and being optionally porous, and or a monosaccharide-polyol, preferably mannitol,
at least two different layers,
at least one hydrophilic polymer preferably selected from polysaccharide or derivatives, and more preferably from starch or derivatives,
and advantageously at least one lipid based material, preferably an amphiphilic compound, more preferably a phospholipid, even more preferably phosphoacylglycerol such as hydrogenated lecithin.

Lipid-Based Material

The inner and/or outer layer(s) may also advantageously include at least one lipid-based material.

According to a particular embodiment of this invention, such a lipid-based material may have amphiphilic properties, that is to say having an apolar part and a polar part.

Such lipid-based material can include at least one or several C₁₂-C₂₂ fatty acid chain(s) such as those selected from stearic acid, palmitic acid, oleic acid, linoleic acid, linolenic acid, etc., and mixtures thereof. Preferably these fatty acids chains are hydrogenated. Eventually, these fatty acid chains may be the apolar part of a lipid-based material.

Such lipid-based material is preferably selected from phospholipids. These phospholipids are preferably selected from phosphoacylglycerol, more preferably selected from lecithins, and are in particular hydrogenated lecithin.

The lipid based material may represent from 0.05 to 5% by weight of the microcapsule, in particular from 0.1 to 1% by weight of microcapsule.

By combining three or more compounds (ex: sugar alcohols, polymers, lipid-based material) in the microcapsule of different hardness and/or water solubility, it is possible to adjust the time required for particle having a high wet point-encapsulated microcapsules to break down on the skin. Thus, according to a preferred embodiment, the multi-layer coating contains at least starch as polymer and at least one lipid-based material, which is preferably lecithin.

According to an advantageous embodiment the microcapsules according to the invention include at least one monosaccharide or its derivative and at least one polysaccharide or its derivatives.

According to a preferred embodiment, the microcapsules include a core comprising a monosaccharide derivative and a coating comprising a polysaccharide (or its derivative) including one type of ose or several type of ose(s), preferably several types of oses.

According to a more preferably embodiment, the microcapsules include a core comprising a monosaccharide polyol, preferably selected from mannitol, erythritol, xylitol, sorbitol, and a coating comprising a polysaccharide (or its derivative) including as ose(s) at least one or more D-Glucose unit(s).

According to a preferred embodiment, the microcapsules additionally include a lipid-based material chosen from phospholipids, advantageously selected from phosphoacylglycerol and in particular from lecithins.

In a particular embodiment, the core contains mannitol, starch polymer and cellulose derivatives and optionally a lipid-based material. In such a case, the starch polymer is the main ingredient i.e. the weight amount of starch is greater than the respective amount of mannitol, cellulose derivative and lipid-based material of the core.

According to a particular embodiment of the invention, the microcapsules comprise at least:
a core comprising at least one particle having a high wet point and being optionally porous, a monosaccharide-polyol, preferably mannitol, a lipid based material preferably lecithin and a hydrophilic polymer preferably starch,
an inner layer comprising starch as a binder, a polymer selected form alkylacrylic/alkylmethacrylic acid copolymers and their derivatives, a lipid based material preferably hydrogenated lecithin, a plasticizer, microcrystalline cellulose, hydroxypropylcellulose and optionally at least one particle having a high wet point, optionally porous, which may be the same or different from the particle having a high wet point, contained in the core,
an outer layer comprising TiO₂, a polymer preferably selected form alkylacrylic/alkylmethacrylic acid copolymers and their derivatives and a optionally a binder preferably starch.

According to another particular embodiment of the invention, the microcapsules comprise at least:
a core comprising at least one particle having a high wet point being optionally porous, a monosaccharide-polyol, preferably mannitol, a lipid based material preferably lecithin and a hydrophilic polymer preferably starch,
an inner layer made of comprising at least one particle having a high wet point being optionally porous, which may be the same or different from the particle having a high wet point, contained in the core, a monosaccharide-polyol, preferably mannitol, a lipid based material preferably hydrogenated lecithin,
an outer layer made of a lipid based material preferably hydrogenated lecithin and a hydrophilic polymer preferably starch.

Particles Having a High Wet Point, which are Optionally Porous

The microcapsule used according to the invention comprises at least 5%, preferably at least 10%, more preferably at least 30%, better at least 40%, even better at least 50%, advantageously at least 60% and in particular between 30 and 80% preferably between 40 and 75% by weight of particle(s) having a high wet point, which is/are optionally porous, relative to the weight of the microcapsule.

The porosity of the encapsulated particles may be characterized by a specific surface area. The porosity character may be observed by microscopy, in particular electronic microscopy.

The microcapsules used according to the invention advantageously have a porosity greater than 100 m²/g, particularly a porosity of from 300 m²/g to 1,500 m²/g according to the BET method.

The BET specific surface area is determined according to the BET (Brunauer-Emmet-Teller) method described in the Journal of the American Chemical Society, vol. 60, page 309, February 1938 and corresponding to the international standard ISO 5794/1 (appendix D). The BET specific surface area corresponds to the total specific surface area (thus including micropores) of the powder.

The encapsulated particles are further defined by their high wet point that is a wet point for oil and/or water equal or greater than 100 ml/100 g preferably greater than 150 ml/100 g.

According to a specific embodiment, the encapsulated particles used according to the invention are fillers.

For the purposes of the present invention, the term “fillers” should be understood as meaning colourless or white solid particles of any form, which usually are in an insoluble and dispersed form in the medium of the composition.

These fillers, of mineral or organic, natural or synthetic nature, give the composition containing them softness and give the makeup result a matt effect and uniformity.

Particles with a High Wet Point for Oil

A microcapsule according to the invention comprises at least one filler with capacity for absorbing and/or adsorbing an oil or a liquid fatty substance, for instance sebum (from the skin), also known as a “sebum-pump filler”.

In particular, the filler used according to the invention has an oil absorption capacity of greater than or equal to 1 ml/g, that is 100 ml/100 g.

This oil-absorbing filler may also advantageously have a BET specific surface area of greater than or equal to 300 m²/g, preferably greater than 500 m²/g and preferentially greater than 600 m²/g, and especially less than 1,500 m²/g.

The filler under consideration is thus characterized in that it has an oil uptake of greater than or equal to 1 ml/g, especially greater than or equal to 1.5 ml/g, especially ranging from 1.5 ml/g to 20 ml/g, or even ranging from 1.5 ml/g to 15 ml/g. It preferably has an oil uptake of greater than or equal to 2 ml/g, especially ranging from 2 ml/g to 20 ml/g, or even ranging from 2 ml/g to 15 ml/g.

This oil uptake, which corresponds to the amount of oil absorbed and/or adsorbed by the filler, may be characterized by measuring the wet point according to the method described below.

Method for Measuring the Oil Uptake of Filler:

The oil uptake of a powder is measured according to the method for determining the oil uptake of a powder as described in standard NF T 30-022. It corresponds to the amount of oil adsorbed onto the available surface of the filler by measuring the wet point.

An amount m (in grams) of powder of between about 0.5 g and 5 g (the amount depends on the density of the powder) is placed on a glass plate and isononyl isononanoate is then added dropwise. After addition of 4 to 5 drops of isononyl isononanoate, the isononyl isononanoate is incorporated into the filler using a spatula, and addition of the isononyl isononanoate is continued until conglomerates of isononyl isononanoate and powder have formed. From this point, the isononyl isononanoate is added one drop at a time and the mixture is then triturated with the spatula. The addition of isononyl isononanoate is stopped when a firm, smooth paste is obtained. This paste must be able to be spread over the glass plate without cracks or the formation of lumps. The volume Vs (expressed in ml) of isononyl isononanoate used is then noted.

The oil uptake corresponds to the ratio Vs/m.

The oil-uptake filler under consideration according to the invention may be of organic or mineral nature.

In particular, the oil-absorbing filler is chosen from silicas, silica silylates (in particular hydrophobic silica aerogels), polyamide powders (in particular Nylon-6), acrylic polymer powders, especially polymethyl methacrylate, polymethyl methacrylate/ethylene glycol dimethacrylate, polyallyl methacrylate/ethylene glycol dimethacrylate or ethylene glycol dimethacrylate/lauryl methacrylate copolymer powders; perlites; magnesium carbonate, silicone filler and mixtures thereof.

A person skilled in the art will select from among the abovementioned materials the filler(s) having an oil uptake of greater than or equal to 1 ml/g, in particular greater than or equal to 1.5 ml/g and preferably greater than or equal to 2 ml/g and which are, in this respect, suitable for use in the invention.

Advantageously, the oil-absorbing powder may be a powder coated with a hydrophobic treatment agent.

The hydrophobic treatment agent may be chosen especially from fatty acids, for instance stearic acid; metal soaps, for instance aluminium dimyristate, the aluminium salt of hydrogenated tallow glutamate; amino acids; N-acylamino acids or salts thereof; lecithin, isopropyl triisostearyl titanate, mineral waxes, and mixtures thereof.

The N-acylamino acids may comprise an acyl group containing from 8 to 22 carbon atoms, for instance a 2-ethylhexanoyl, caproyl, lauroyl, myristoyl, palmitoyl, stearoyl or cocoyl group. The salts of these compounds may be aluminium, magnesium, calcium, zirconium, zinc, sodium or potassium salts. The amino acid may be, for example, lysine, glutamic acid or alanine.

The term “alkyl” mentioned in the compounds cited above especially denotes an alkyl group containing from 1 to 30 carbon atoms and preferably containing from 5 to 16 carbon atoms.

Examples of fillers in accordance with the invention, i.e. having an oil uptake of greater than or equal to 1 ml/g and in particular 1.5 ml/g, are described below, with their oil uptake value measured according to the protocol described previously.

In particular, the oil-absorbing filler is chosen from porous silica microspheres, polydimethylsiloxane-coated amorphous silica microspheres, silica silylate powders, amorphous hollow silica particles, precipitated silica powders surface-treated with a mineral wax, porous polymethyl methacrylate/ethylene glycol dimethacrylate spheres, ethylene glycol dimethacrylate/lauryl methacrylate copolymer powders, the hollow PMMA spheres, Nylon-6 powder, Nylon® 12, perlite powders, magnesium carbonate powders, organopolysiloxane powders, preferably coated with silicone resin; hollow hemispherical particles of silicone, hollow hemispherical particles of silicone.

Silica Powders that May be Mentioned Include:
porous silica microspheres, especially those sold under the names Sunsphere® H53 and Sunsphere® H33 (oil uptake equal to 3.70 ml/g); Sunsphere® H51 by the company Asahi Glass and Silica Beads SB 700 Myochi (oil uptake equal to 1.33 ml/g); MSS-500-3H by the company Kobo;
polydimethylsiloxane-coated amorphous silica microspheres, especially those sold under the name SA Sunsphere® H33 (oil uptake equal to 2.43 ml/g);
silica silylate powders, especially those sold under the name Dow Corning VM-2270 Aerogel Fine Particles by the company Dow Corning (oil uptake equal to 10.40 ml/g);
amorphous hollow silica particles, especially those sold under the name Silica Shells by the company Kobo (oil uptake equal to 5.50 ml/g);
precipitated silica powders surface-treated with a mineral wax, such as precipitated silica treated with a polyethylene wax, and especially those sold under the name Acematt OR 412 by the company Evonik-Degussa (oil uptake equal to 3.98 ml/g).

Acrylic Polymer Powders that May be Mentioned Include:
porous polymethyl methacrylate/ethylene glycol dimethacrylate spheres sold under the name Microsponge 5640 by the company Cardinal Health Technologies (oil uptake equal to 1.55 ml/g);
ethylene glycol dimethacrylate/lauryl methacrylate copolymer powders, especially those sold under the name Polytrap® 6603 from the company Dow Corning (oil uptake equal to 6.56 ml/g);
the hollow PMMA spheres sold under the name Covabead® LH 85 by Wacker (oil uptake equal to 1.23 ml/g);
crosslinked poly methyl methacrylate hemispheres (size: 5-20 MICRONS) sold under the commercial name MICROPEARL M310 by MATSUMOTO YUSHI-SEIYAKU;
ethyleneglycol dimethacrylate and methyl methacrylate copolymer (size 6-10 MICRONS) sold under the commercial name TECHPOLYMER MBP-8 by SEKISUI PLASTICS;
acrylates/ethylhexylacrylate copolymer (size 12-18 MICRONS) sold under the commercial name TECHPOLYMER ACP8C by SEKISUI PLASTICS).

Polyamide Powders that May be Mentioned Include:
Nylon-6 powder, especially the product sold under the name Pomp610 by the company UBE Industries (oil uptake equal to 2.02 ml/g);
Nylon® 12, including those sold under the name Orgasol 2002® (oil uptake equal to 1.11 ml/g).

A perlite powder that may especially be mentioned is the product sold under the name Optimat 2550 OR by the company World Minerals (oil uptake equal to 2.4 ml/g).

A magnesium carbonate powder that may especially be mentioned is the product sold under the name Tipo Carbomagel by the company Buschle & Lepper (oil uptake equal to 2.14 ml/g).

A silicone filler may be chosen from:
organopolysiloxane powders, preferably coated with silicone resin;
hollow hemispherical particles of silicone;

and a mixture thereof.

In a preferred embodiment, the silicone filler is an organopolysiloxane powder, preferably coated with silicone resin.

The hollow hemispherical particles of silicone may be NLK 500, NLK 506 and NLK 510 from Takemoto Oil and Fat. In particular, mention may be made especially of NLK 506 (oil uptake equal to 1.66 ml/g).

The oil-absorbing filler that is particularly preferred is a silica powder and more particularly a silica powder with an oil uptake at least equal to 3.70 ml/g, and especially the products sold under the name Sunsphere® H33 by the company Asahi Glass and under the name Dow Corning VM-2270 Aerogel Fine Particles by the company Dow Corning.

Aerogels may be particularly mentioned as preferred oil absorbing fillers used in the microcapsules of the present invention.

The hydrophobic aerogels used in the microcapsules of present invention may be organic, inorganic or organic-inorganic hybrid aerogels.

The organic aerogels may be based on resins from among the following: polyurethanes, resorcinol-formaldehyde, polyfurfuranol, cresol-formaldehyde, phenol-furfuranol, polybutadiene, melamine-formaldehyde, phenol-furfural, polyimides, polyacrylates, polymethacrylates, polyolefins, polystyrenes, polyacrylonitriles, phenol-formaldehyde, polyvinyl alcohol, dialdehydes, polycyanides, epoxys, celluloses, cellulose derivatives, chitosan, agar, agarose, alginate, starches, and mixtures thereof. Aerogels based on organic-inorganic hybrids, for example silica-PMMA, silica-chitosan and silica-polyether, are also envisaged. Patent applications US 2005/0 192 366 and WO 2007/126 410 describe such organic-inorganic hybrid materials.

The sizes of the aerogel particles used in the microcapsules according to the invention can be measured by static light scattering using a commercial particle size analyser such as the MasterSizer 2000 machine from Malvern. The data are processed on the basis of the Mie scattering theory. This theory, which is exact for isotropic particles, makes it possible to determine, in the case of non-spherical particles, an “effective” particle diameter. This theory is especially described in the publication by Van de Hulst, H. C., “Light Scattering by Small Particles”, Chapters 9 and 10, Wiley, New York, 1957.

According to an advantageous embodiment, the hydrophobic aerogel particles used in the microcapsules of the present invention have a specific surface area per unit of mass (SM) ranging from 600 to 800 m²/g and a size, expressed as the volume-mean diameter (D[0.5]), ranging from 5 to 20 μm and better still from 5 to 15 μm.

The hydrophobic aerogel particles used in the microcapsules of the present invention may advantageously have a tapped density p ranging from 0.02 g/cm³ to 0.10 g/cm³ and preferably from 0.03 g/cm³ to 0.08 g/cm³. In the context of the present invention, this density may be assessed according to the following protocol, known as the tapped density protocol:

40 g of powder are poured into a measuring cylinder; the measuring cylinder is then placed on the Stav 2003 machine from Stampf Volumeter; the measuring cylinder is subsequently subjected to a series of 2,500 tapping actions (this operation is repeated until the difference in volume between 2 consecutive tests is less than 2%); and then the final volume Vf of tapped powder is measured directly on the measuring cylinder. The tapped density is determined by the ratio m/Vf, in this instance 40/Vf (Vf being expressed in cm³ and m in g).

According to one embodiment, the hydrophobic aerogel particles used in the microcapsules of present invention have a specific surface area per unit of volume Sv ranging from 5 to 60 m²/cm³, preferably from 10 to 50 m²/cm³ and better still from 15 to 40 m²/cm³.

The specific surface area per unit of volume is given by the relationship: Sv=SM−p where p is the tapped density expressed in g/cm³ and SM is the specific surface area per unit of mass expressed in m²/g, as defined above.

Preferably, the hydrophobic aerogel particles used in the microcapsules of the invention have an oil-absorbing capacity, measured at the wet point, ranging from 5 to 18 ml/g, preferably from 6 to 15 ml/g and better still from 8 to 12 ml/g.

According to a particular embodiment, the aerogel particles used are inorganic and are more particularly hydrophobic silica aerogel particles having the properties stated previously.

Silica aerogels are porous materials obtained by replacing (especially by drying) the liquid component of a silica gel with air.

They are generally synthesized via a sol-gel process in a liquid medium and then dried, usually by extraction with a supercritical fluid, the one most commonly used being supercritical CO2. This type of drying makes it possible to avoid shrinkage of the pores and of the material. The sol-gel process and the various drying operations are described in detail in Brinker C. J. and Scherer G. W., Sol-Gel Science, New York: Academic Press, 1990.

The hydrophobic silica aerogels used in the microcapsules of the present invention are preferably silylated silica aerogels (INCI name: silica silylate).

The term “hydrophobic silica” means any silica whose surface is treated with silylating agents, for example halogenated silanes such as alkylchlorosilanes, siloxanes, in particular dimethylsiloxanes such as hexamethyldisiloxane, or silazanes, so as to functionalize the OH groups with silyl groups Si—Rn, for example trimethylsilyl groups. As regards the preparation of hydrophobic silica aerogel particles that have been surface-modified by silylation, reference may be made to document U.S. Pat. No. 7,470,725.

Use will in particular be made of the hydrophobic silica aerogel particles that have been surface-modified with trimethylsilyl groups. As hydrophobic silica aerogels that may be used in the invention, examples that may be mentioned include the aerogel sold under the name VM-2260 (INCI name: Silica silylate) by Dow Corning, the particles of which have a mean size of about 1000 microns and a specific surface area per unit of mass ranging from 600 to 800 m²/g.

Mention may also be made of the aerogels sold by Cabot under the references Aerogel TLD 201, Aerogel OGD 201 and Aerogel TLD 203, Enova® Aerogel MT 1100 and Enova Aerogel MT 1200.

Use will also be made of the aerogel sold under the name Enova® Aerogel MT 1100 (INCI name: Silica silylate) by Cabot, the particles of which have a mean size ranging from 2-25 microns and a specific surface area per unit of mass ranging from 600 to 800 m²/g.

As already explained, use will more particularly be made of the aerogel sold under the name VM-2270 (INCI name: Silica silylate) by Dow Corning, the particles of which have a mean size ranging from 5-15 microns and a specific surface area per unit of mass ranging from 600 to 800 m²/g.

Particles with a High Wet Point for Water

Similarly, the term “wet point for water” in the specification means a quantity or amount of water which is necessary to make a target powder completely wet, which can be recognized, in particular, by the formation of a paste with the target powder.

The particle used in the microcapsules according to the present invention has a wet point for water being at least 100 ml/100 g, preferably ranging from 100 to 600 ml/100 g and more preferably from 150 to 500 ml/100 g.

The wet point for water can be determined by the following protocol.

(1) 2 g of a target powder is kneaded with a spatula on a glass plate while adding water with a density of 0.998 g/ml.

(2) When the target powder becomes completely wet and starts to form a paste, the weight of the added water is determined as the weight of wet point.

(3) The wet point for water is calculated from the equation: Wet point for water (ml/100 g)={(the weight of wet point)/2 g}×100/the density of water.

As encapsulated particle with a high wet point for water, mention may be made of spherical cellulose particles, for example, the following ones marketed by Daito Kasei in Japan:

Cellulobeads USF (wet point for oil is 296.0 ml/100 g, wet point for water is 400.8 ml/100 g, the ratio of the wet point for water/the wet point for oil is 1.4) with a particle size of 4 μm;

Cellulobeads D-5 (wet point for oil is 49.8 ml/100 g, wet point for water is 205.0 ml/100 g, the ratio of the wet point for water/the wet point for oil is 4.1) with a particle size of 10 μm;

Cellulobeads D-10 (wet point for oil is 44.0 ml/100 g, wet point for water is 164.0 ml/100 g, the ratio of the wet point for water/the wet point for oil is 3.7) with a particle size of 15 μm;

MOISCELL PW D-5 XP (wet point for oil is 58.6 ml/100 g, wet point for water is 281.5 ml/100 g, the ratio of the wet point for water/the wet point for oil is 4.8) with a particle size of 10 μm (potassium succinate cellulose); and

MOISCELL PW D-50 XP (wet point for oil is 39.9 ml/100 g, wet point for water is 160.0 ml/100 g, the ratio of the wet point for water/the wet point for oil is 4) with a particle size of 50 μm (potassium succinate cellulose).

Cellulobeads USF and Cellulobeads D-5 are preferable. Cellulobeads USF are most preferable.

II. Methods for Preparing Microcapsules

The microcapsules may be produced by several methods known to the man skilled in the art within the coating or encapsulation domain, including spray drying, pelletization, granulation, coating, etc.

For example, the method includes:

preparing an aqueous solution containing water, a lower alcohol such as ethanol, and a hydrophilic gelling agent which is soluble in water and the alcohol,

dispersing an aerogel and optionally a pigment in the aqueous solution; and

coating a core with the aqueous solution.

The solution for coating the core may not contain water. For example, the solution can contain the lower alcohol and the hydrophilic gelling agent without water.

The hydrophilic gelling agent can be any one or combination of those known in the art which are soluble in water and a lower alcohol such as ethanol. Preferably, hydroxypropylmethyl cellulose (HPMC) can be used as the hydrophilic gelling agent. The aerogel can be any one or combination of those listed above, for example, hydrophobic silica aerogel particles. The pigment can be, for example, a particle of high density chosen among filler, nacres and their mixtures. Preferably, a natural pigment such as Perlite or a pearl pigment such as Timica Terra White MN4501 can be used.

The hydrophobic silica aerogel particle can be any one or combination of those listed above.

Preferably the microcapsules are produced by this process and comprise a combination of hydrophobic silica aerogel particle, and a particle of high density chosen among filler, nacres and their mixtures.

The hydrophobic silica aerogel particles have a specific surface area per unit of mass (SM) ranging from 500 to 1500 m²/g, preferably from 600 to 1200 m²/g and better still from 600 to 800 m²/g, and advantageously a size expressed as the volume-mean diameter (D[0.5]) ranging from 1 to 1500 μm, preferably from 1 to 1000 μm, more preferentially from 1 to 100 μm, in particular from 1 to 30 μm, more preferably from 5 to 25 μm, better still from 5 to 20 μm and even better still from 5 to 15 μm.

Said hydrophobic aerogel particles have preferably an oil absorption capacity, measured at the wet point, ranging from 5 to 18 ml/g, preferably from 6 to 15 ml/g and better still from 8 to 12 ml/g of particles.

Advantageously said hydrophobic aerogel particles have a tapped density ranging from 0.02 g/cm³ to 0.10 g/cm³ and preferably from 0.03 g/cm³ to 0.08 g/cm³.

According to a specific embodiment, the hydrophobic silica aerogel particles are hydrophobic silica aerogel particles that are surface modified with trimethylsilyl groups, preferably hydrophobic silica aerogel particles having the INCI name Silica silylate.

Preferably the particle of high density is a lamellar particle, more preferably chosen among mica, perlite, sericite, kaolin, talc and silica, nacres and mixtures thereof.

The particle of high density may also be a spheric particle more preferably chosen among organic fillers.

The filler can be chosen from Perlite-MSZ12 and Timica Terra White MN4501.

In a preferred embodiment, the composition according to the invention comprise, as a particle having a high wet point, a hydrophobic silica aerogel particle which is present in the core and/or in at least one inner layer.

Advantageously the core and/or at least one inner layer containing the hydrophobic silica aerogel particle further comprise at least one particle of high density chosen among filler, nacres and their mixtures, preferably the particle of high density is a lamellar particle, more preferably chosen among mica, perlite, sericite, kaolin, talc and silica, nacres and mixtures thereof.

The amount of each of water, the alcohol, the hydrophilic gelling agent, the aerogel, the pigment, and the core can be any amount determined by a person of ordinary skill in the art. For example, 25-75 weight parts of the hydrophilic gelling agent is added to a mixture of 500-1,500 weight parts of water and 2,000-5,000 weight parts of the alcohol, and 100-300 weight parts of the aerogel and 200-400 weight parts of the pigment are added thereto. For example, 300-600 g of the core is coated with the coating solution. Preferably, the aqueous solution contains 0-20 wt % of water and 80-100 wt % of the lower alcohol.

The coating step can be carried out with a spray drying process.

Spray drying processes may be carried out by any method e.g. tangential, bottom or top spray drying. It may also be combined with a drying in a fluidized bed process. These alternatives may further be combined in order to obtain microcapsules having the required properties.

Preferably at least one outer layer, more preferably all outer layers are obtained by a combination of one or several of these alternatives: tangential, bottom or top spray drying optionally combined with a fluidized bed process.

For example, the microcapsules may be obtained by a method comprising mixture of the compounds (particles having a high wet point, other optional actives, polymers, solvents) and drying to form capsules as disclosed in WO01/35933 and WO2011/027960, or a method comprising granulation and coating by spray drying as disclosed in FR2841155, or by fluidized bed technology, which has been used in the food and pharmaceutical industry for a long time for coating and encapsulating ingredients. As an example may be cited WO2008/139053, which concerns the preparation of spheroid multilayer capsules comprising a core of sugar and concentric layers of pharmaceutical actives. Fixation of pharmaceutical actives on the core is achieved by impregnation, pulverization or projection, and then the 1^{st} layer is dried before application of a second one.

Fluid Bed Process

Fluid bed process is disclosed for example in Teunou et al. (Fluid-Bed Coating, Poncelet, 2005, D. Food Science and Technology (Boca Raton, Fla., United States), Volume 146 Issue Encapsulated and Powdered Foods, Pages 197-212). A specific feature of the fluid bed process is that it leads to coated particles wherein the core is well encapsulated, compared to spray drying, which leads to a matrix with the core material randomly dispersed in a polymer.

In a preferred embodiment, the microcapsules are obtained by fluid bed process.

According to this embodiment, preferably at least one layer of the microcapsules is obtained by fluid bed process.

In a particular embodiment, the outer layer is obtained by fluid bed process.

In another particular embodiment at least one inner layer is obtained by fluid process.

At least one layer, most preferably, all layers are obtained by fluid bed process.

The man skilled in the art knows how to adjust air quantity, liquid quantity and temperature allowing to reproduce a microcapsule according to the invention.

Preferably a fluid bed process implemented according to the invention includes Wurster process and/or tangential spray process. Such a process allows, contrary to a pelletization process, to prepare spherical capsules with a core surrounded by one or more circumferential layers.

When the whole process for preparing the layers surrounding the core of the microcapsules according to the invention is carried out by fluid bed process, the microcapsule layers are advantageously regular, concentric and present a homogenous thickness.

Advantageously this water acts as a swelling agent or as a softening agent towards these microcapsules without breaking them. The microcapsules are not inert when placed in water either they swell: their diameter significantly increases with an optional softening of the microcapsules, or the microcapsules significantly soften without increasing of the diameter, they become more malleable and easier to break when applied onto the skin.

Water is able to act on the softening kinetics of the microcapsules and more particularly it allows to obtain a good balance between softening kinetics and hardness.

As a consequence, water is particularly advantageous for softening these microcapsules suitable for the present invention, in an appropriate way, since it plays a role on softening kinetics of the microcapsules.

The microcapsules are preferably deformable in the presence of an aqueous phase, notably in the presence of water.

According to this embodiment of the invention, composition comprise water in a content ranging from 30% to 99% by weight, preferably from 40% to 95% more preferably from 50% to 90% by weight relative to the total weight of the composition.

Optionally it also comprises at least one compound chosen from polyols, glycols and C₂-C₈ monoalcohols, and mixtures thereof.

The polyol is preferably selected from the group consisting in glycerol, glycols, preferably propylene glycol, butylene glycol, pentylene glycol, hexylene glycol, dipropylene glycol, diethylene glycol, glycol ethers, preferably mono-, di- or tripropylene glycol of alkyl(C₁-C₄)ether or mono-, di- or triethylene glycol of alkyl(C₁-C₄)ether, and mixtures thereof.

Compositions according to this embodiment are advantageously in the form of an oil-in-water emulsion.

The invention is illustrated in greater detail by the examples according to the invention described below. Unless otherwise mentioned, the amounts indicated are expressed as mass percentages of active material.

### EXAMPLES

Some examples of the present invention are provided below. These examples are illustrative, but not limiting the scope of the present invention. Reasonable variations can be made herein without departing from the scope of the present invention.

Different examples of preparation of microcapsules according to the invention are here below described for illustrating the invention.

### Example 1a

STP-F Seed 4050 (a core sphere comprising 20-30% mannitol; 20-30% microcrystalline cellulose; and 40-50% corn starch) is used as core.

To a mixed solution of 750 g of water and 3000.0 g of ethanol, 50.0 g of HPMC (Hydroxyl propyl methyl cellulose) is added and completely dissolved at room temperature. To the resulting mixture, 150 g of silica silylate aerogel (Dow Corning® VM-2270 AEROGEL FINE PARTICLES; aerogel at 98% dry matter in water), and 350 g of Perlite-MSZ12 are added and well and dispersed with a homogenizer at 3000 rpm for 20 min to prepare a first charged coating solution.

450.0 g of STP-F Seed 4050 is introduced into a fluidized bed coating system (Glatt GPCG 1, tangential spray) as a core seed and subjected to a coating at 450˜500 ml/h of feeding rate of inner layer charged solution to obtain particles having a “STP-F Seed 4050” core coated with a charged layer.

Coated particles prepared according to this process are generated having a size range of approximately 355 μm˜600 μm.

### Example 1b

STP-F Seed 4050 is used as core.

To a mixed solution of 1200.0 g of water and 4800.0 g of ethanol, 50.0 g of HPMC (Hydroxyl propyl methyl cellulose) are added and completely dissolved at room temperature. To the resulting mixture, 250 g of silica silylate aerogel (Dow Corning® VM-2270 AEROGEL FINE PARTICLES; aerogel at 98% dry matter in water), and 250 g of Timica Terra White MN4501 are added and well dispersed with a homogenizer at 3000 rpm and 20 min to prepare 1st. layer charged coating solution.

450.0 g of STP-F Seed 4050 is introduced into a fluidized bed coating system (Glatt GPCG 1, tangential spray) as a core seed and subjected to a coating at 500 ml/h of feeding rate of inner layer charged solution to obtain particles having a “STP-F Seed 4050” core coated with charged layer.

Coated particles prepared according to this process are generated having a size range of approximately 355 μm˜600 μm.

### Example 2

STP-F Seed 4050 (which is a core with fluid bed process by KPT) is used as core.

To a mixed solution of 1200.0 g of water and 4800.0 g of ethanol, 50.0 g of HPMC (Hydroxyl propyl methyl cellulose) are added and completely dissolved at room temperature. To the resulting mixture, 150 g of VM-2270 AEROGEL FINE PARTICLES, 350 g of Timica Terra White MN4501 are added and well dispersed with a homogenizer at 3000 rpm and 20 min to prepare 1st. layer charged coating solution.

450.0 g of STP-F Seed 4050 is introduced into a fluidized bed coating system (Glatt GPCG 1, tangential spray) as a core seed and subjected to a coating at 500 ml/h of feeding rate of inner layer charged solution to obtain particles having a STAPHERE F Seed 4050′ core coated with a charged layer.

Coated particles prepared according to this process are generated having a size range of approximately 355 μm˜600 μm.

The following particles according to the invention are implemented in the examples:
silica (AMORPHOUS SILICA MICROSPHERES (5 μm)), entitled “B” in the following examples
SUNSPHERE H 51 sent by AGC SI-TECH, entitled “C” in the following examples
PMMA (HOLLOW SPHERES CREUSES OF POLY METHYL METHACRYLATE (10 MICRONS) sent by SENSIENT, entitled “D” in the following examples
Cellulose (CELLULOBEADS USF sent by KOBO), entitled “E” in the following examples,
Aerogel at 98% dry matter in water (DOW CORNING VM-2270 AEROGEL FINE PARTICLES sent by DOW CORNING), entitled “F” in the following examples

### Example 3

By using the ingredients and contents described in the below table, a microcapsule having a core and 2 layers is prepared by the procedure provided in Example 1 or 2:

(1) particle having a high wet point C

(2) Ingredients: Core seed—porous particle inner layer—TiO₂ particle layer

| | | |
|---|---|---|
| Core | Mannitol | 16.45% |
| 1^{st} layer | particle having a high | 50.0% |
| | wet point C | |
| | Lecithin | 0.5 |
| | Corn Starch binder | 2.0% |
| 2^{nd} layer | Titanium dioxide | qsp. 100% |
| | Lecithin | 0.2% |
| | Corn Starch binder | 0.8% |

Percentages indicate weight percent relative to the total microcapsule weight.

### Example 4

By using the ingredients and contents described in the below table, a microcapsule having a core and 3 layers is prepared by the procedure provided in Example 1 or 2:

(1) particle having a high wet point D

(2) Ingredients: Core seed—particle inner layer—TiO₂ particle layer—outer color layer

| | | |
|---|---|---|
| Core | Mannitol | 6.5% |
| 1^{st} layer | particle having a | 17.8% |
| | high wet point D | |
| | Sunpuro Yellow | 2.00% |
| | Lecithin | 5.0% |
| | Eudragit RSPO | 4.0% |
| 2^{nd} layer | Titanium dioxide | qsp. 100% |
| | Lecithin | 5.0% |
| | Eudragit RSPO | 4.0% |
| 3^{rd} layer | D&C Red30 | 0.8% |
| | Cornstarch binder | 0.4% |

Percentages indicate weight percent relative to the total microcapsule weight.

### Example 5

By using the ingredients and contents described in the below table, a microcapsule having a core and 2 layers is prepared by the procedure provided in Example 1 or 2:

(1) particle having a high wet point D

(2) Ingredients: Core seed—particle inner layer—TiO₂ particle layer

| | | |
|---|---|---|
| Core | Mannitol | 17.8% |
| 1^{st} layer | particle having a | 19.8% |
| | high wet point D | |
| | Lecithin | 0.2% |
| | Corn Starch binder | 0.8% |
| 2^{nd} layer | Titanium dioxide | qsp. 100% |
| | Mannitol | 5.0% |
| | Corn Starch | 5.0% |
| | Lecithin | 0.3% |
| | Corn Starch binder | 1.2% |

Percentages indicate weight percent relative to the total microcapsule weight.

### Example 6

By using the ingredients and contents described in the below table, a microcapsule having a core and 2 layers is prepared by the procedure provided in Example 1 or 2:

(1) Ingredients: Core seed—particle inner color layer—TiO₂ particle layer

| | | |
|---|---|---|
| Core | Mannitol | 13.7% |
| 1^{st} layer | particle having a | 21.64% |
| | high wet point E | |
| | Lecithin | 0.20% |
| | Corn Starch Binder | 1.0% |
| 2^{nd} layer | Titanium dioxide | qsp. 100% |
| | Lecithin | 0.3% |
| | Corn Starch Binder | 1.5% |

Percentages indicate weight percent relative to the total microcapsule weight.

### Example 7

By using the ingredients and contents described in the below table, a microcapsule having a core and 3 layers is prepared by the procedure provided in Example 1 or 2:

(1) particle having a high wet point E

(2) Ingredients: Core seed—particle inner layer—TiO₂ particle layer—Outer color layer

| | | |
|---|---|---|
| Core | Mannitol | 16.81% |
| 1^{st} layer | particle having a | 49.15% |
| | high wet point E | |
| | Lecithin | 0.29% |
| | Corn Starch Binder | 1.97% |
| 2^{nd} layer | Titanium dioxide | qsp 100% |
| | Lecithin | 0.1% |
| | Corn Starch Binder | 0.49% |
| 3^{rd} layer | Sunpuro Yellow | 1.0% |
| | Sunpuro Red | 0.2% |
| | Corn Starch Binder | 0.5% |

Percentages indicate weight percent relative to the total microcapsule weight.

### Example 8

By using the ingredients and contents described in the below table a microcapsule having a core and 3 layers is prepared by the procedure provided in Example 1 or 2:

(1) particle having a high wet point F

(2) Ingredients: Core seed—particle inner layer—TiO₂ particle layer—Outer color layer

| | | | | |
|---|---|---|---|---|
| Core | Organic core | 4.0% | Cellulose | 1.12% |
| | | | Mannitol | 1.0% |
| | | | Zea Mays(corn) starch | 1.84% |
| | | | Hydrogenated Lecithin | 0.04% |
| 1^{st} layer | particle having a | 55.0% | particle having a | 55 |
| | high wet point F | | high wet point F | |
| | Lecithin | 0.50% | Hydrogenated Lecithin | 0.50% |
| | Mannitol | 3.5% | Mannitol | 3.5% |
| | Corn Starch Binder | 2.0% | Zea Mays(corn) starch | 2.0% |
| 2^{nd} layer | Titanium dioxide | qsp 100%. | Titanium dioxide | qsp 100%. |
| | Corn Starch | 3.62% | Zea Mays(corn) starch | 3.62% |
| | Cellulose | 9.0% | Cellulose | 9.0% |
| | Mannitol | 13.0% | Mannitol | 13.0% |
| | Lecithin | 0.25% | Hydrogenated Lecithin | 0.25% |
| | Corn Starch Binder | 1.8% | Zea Mays(corn) starch | 1.8% |
| 3^{rd} Layer | Satin White | 1.8% | Synthetic Fluorphlogopite | 1.035% |
| | | | Tin oxide | 0.009% |
| | | | Titanium Dioxide | 0.756% |
| | D&C Red30 | 0.03% | Red30 Al. Lake | 0.03% |
| | Corn Starch Binder | 0.5% | Zea Mays(corn) starch | 0.5% |

Percentages indicate weight percent relative to the total microcapsule weight.

### Example 9

By using the ingredients and contents described in the below table, a microcapsule having a core and 3 layers is prepared by the procedure provided in Example 1 or 2:

(1) particle having a high wet point C

(2) Ingredients: Core seed—particle inner layer—TiO₂ particle layer—Outer color layer

| | | |
|---|---|---|
| Core | Organic core | 34.4% |
| 1^{st} layer | particle having a | 50.0% |
| | high wet point C | |
| | Lecithin | 0.50% |
| | Mannitol | 4.0% |
| | Corn Starch Binder | 2.0% |
| 2^{nd} layer | Titanium dioxide | qsp 100% |
| | Lecithin | 0.1% |
| | Corn Starch Binder | 0.4% |
| 3^{rd} Layer | C. Monarch gold | 3.0% |
| | Corn Starch Binder | 0.6% |

Percentages indicate weight percent relative to the total microcapsule weight.

(3) Ingredient of each layer (in details):

| | | | | |
|---|---|---|---|---|
| Core | Organic core | 34.4% | Zea Mays(corn) Starch | 14.3% |
| | | | Mannitol | 10.5% |
| | | | Cellulose | 9.6% |
| 1^{st} layer | particle having a high | 50.0% | particle having a high wet | 50 |
| | wet point D | | point D | |
| | Lecithin | 0.50% | Hydrogenated Lecithin | 0.50% |
| | Mannitol | 4.0% | Mannitol | 4.0% |
| | Corn Starch Binder | 2.0% | Zea Mays(corn) Starch | 2.0% |
| 2^{nd} layer | Titanium dioxide | qsp. 100% | Titanium dioxide | qsp. 100% |
| | Lecithin | 0.1% | Hydrogenated Lecithin | 0.1% |
| | Corn Starch Binder | 0.4% | Zea Mays(corn) Starch | 0.4% |
| 3^{rd} Layer | C. Monarch gold | 3.0% | Mica | 1.575% |
| | | | Titanium Dioxide | 1.29% |
| | | | Iron oxide Red | 0.12% |
| | | | Tin Oxide | 0.015% |
| | Corn Starch Binder | 0.6% | Zea Mays(corn) Starch | 0.6% |

Percentages indicate weight percent relative to the total microcapsule weight.

### Example 10

By using the ingredients and contents described in the below table, a microcapsule having a core and 2 layers is prepared by the procedure provided in Example 1 or 2:

(1) Ingredients: Core seed—particle layer—Outer color layer

| | | |
|---|---|---|
| Core | Mannitol | 27.85% |
| 1^{st} layer | particle having a | qsp. 100% |
| | high wet point B | |
| | Lecithin | 0.5% |
| | Corn Starch Binder | 1.5% |
| 2^{nd} layer | D&C Red30 | 0.145% |
| | Satin White | 4.55% |
| | Corn Starch Binder | 0.3% |

Percentages indicate weight percent relative to the total microcapsule weight.

(2) Ingredient of each layer (in details):

| | | | | |
|---|---|---|---|---|
| Core | Mannitol | 27.85% | Mannitol | 27.85% |
| 1^{st} layer | particle having a high | qsp. | particle having a high wet | qsp. 100% |
| | wet point D | | point D | |
| | Lecithin | 0.5% | Lecithin | 0.5% |
| | Corn Starch Binder | 1.5% | Corn Starch Binder | 1.5% |
| 2^{nd} layer | D&C Red30 | 0.145% | D&C Red30 | 0.145% |
| | Satin White | 4.55% | Synthetic Fluorphlogopite | 2.66% |
| | | | Tin oxide | 0.023% |
| | | | Titanium Dioxide | 1.867% |
| | Corn Starch Binder | 0.3% | Corn Starch Binder | 0.3% |

Percentages indicate weight percent relative to the total microcapsule weight.

### Example 11

By using the ingredients and contents described in the below table, a microcapsule having a core and 3 layers is prepared by the procedure provided in Example 1 or 2:

(1) reflective particle

(2) Ingredients: Core seed—particle inner layer—TiO₂ particle layer—Outmost shell

| | | | | |
|---|---|---|---|---|
| Core | Organic core | 4.0% | Cellulose | 1.0% |
| | | | Mannitol | 1.0% |
| | | | Zea Mays(corn) Starch | 2.0% |
| 1^{st} layer | particle having a high | 50.0% | particle having a high wet | 50% |
| | wet point F | | point F | |
| | Lecithin | 0.50% | Hydrogenated Lecithin | 0.50% |
| | Mannitol | 3.5% | Mannitol | 3.5% |
| | Corn Starch Binder | 2.0% | Zea Mays(corn) Starch | 2.0% |
| 2^{nd} layer | Titanium dioxide | qsp. 100% | Titanium dioxide | qsp. 100% |
| | Corn Starch | 2.0% | Zea Mays(corn) Starch | 2.0% |
| | Cellulose | 5.0% | Cellulose | 5.0% |
| | Mannitol | 6.5% | Mannitol | 6.5% |
| | Lecithin | 0.25% | Hydrogenated Lecithin | 0.25% |
| | Corn Starch Binder | 1.0% | Zea Mays(corn) Starch | 1.0% |
| 3^{rd} Layer | Iron oxide Red | 0.05% | Iron oxide Red | 0.05% |
| | Iron oxide Yellow | 0.01% | Iron oxide Yellow | 0.01% |
| | Cellulose | 5.0% | Cellulose | 5.0% |
| | Mannitol | 6.5% | Mannitol | 6.5% |
| | Corn Starch | 7.44% | Zea Mays(corn) Starch | 7.44% |
| | Lecithin | 0.25% | Hydrogenated Lecithin | 0.25% |
| | Corn Starch Binder | 1.0% | Zea Mays(corn) Starch | 1.0% |

Percentages indicate weight percent relative to the total microcapsule weight.

### Example 12

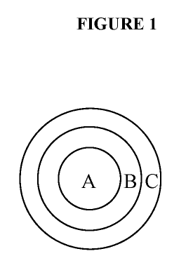

By using the ingredients and contents described in the below table, a microcapsule, as shown in FIG. 1, having a core including notably mannitol and Particle having a high wet point, for instance F, is prepared by the procedure provided in Example 1 or 2:

| | | | | |
|---|---|---|---|---|
| Core | Lecithin | 0.9% | Hydrogenated Lecithin | 0.9% |
| | Mannitol | 18.9% | Mannitol | 18.9% |
| | Corn Starch Binder | 4.5% | Zea Mays(corn) Starch | 4.5% |
| | Particle having a high | 75.6% | Particle having a high wet | 75.6% |
| | wet point F | | point F | |
| 1^{st} layer | Particle having a high | 60.0% | Particle having a high wet | 60.0 |
| | wet point F | | point F | |
| | Lecithin | 0.04% | Hydrogenated Lecithin | 0.040% |
| | Mannitol | 15.0% | Mannitol | 15.0% |
| | Corn Starch Binder | 0.20% | Zea Mays(corn) Starch | 0.20% |
| 2^{nd} layer | Lecithin | 0.01% | Hydrogenated Lecithin | 0.01% |
| | Corn Starch Binder | 0.025% | Zea Mays(corn) Starch | 0.025% |

We claim:

1. A composition for cosmetic raw material comprising, at least one microcapsule containing at least one encapsulated releasable material, wherein the microcapsule comprising at least one core and at least one layered coating surrounding the core, and the encapsulated material is at least one particle having a high wet point and is optionally porous, and being only released from the microcapsule(s) when the composition is applied onto a keratin material, such as keratin fibers or skin.

2. The composition of claim 1, wherein the particle(s) having a high wet point is (are) porous.

3. The composition of claim 1, wherein the microcapsule comprises at least 5%, preferably at least 10%, more preferably at least 30%, better at least 40%, even better at least 50%, advantageously at least 60% and in particular between 30 and 80% preferably between 40 and 75% by weight of the particle(s) relative to the weight of the microcapsule.

4. The composition of claim 1, comprising,
microcapsules containing releasable material(s), the microcapsules comprising:
a core comprising at least one of the particle(s) and optionally at least one organic material,
at least one layered coating surrounding the core, the layered coating comprising a binder selected from at least one polymer, at least one lipid-based material, and their mixture, preferably their mixture and optionally at least one particle having a high wet point, the particle being optionally porous, which may be the same or different from the particle having a high wet point contained in the core, and
an outer layer comprising a hydrophilic polymer.

5. The composition of claim 1, wherein the core comprises at least one monosaccharide or its derivatives as the organic material, in particular a monosaccharide-polyol advantageously selected from mannitol, erythritol, xylitol, sorbitol and mixtures thereof, preferably mannitol.

6. The composition of claim 1, wherein the layered coating surrounding the core comprises at least one hydrophilic polymer(s) selected from the group consisting of:
acrylic or methacrylic acid homopolymers or copolymers or salts and esters thereof;
copolymers of acrylic acid and of acrylamide and its salts and esters thereof;
polyhydroxycarboxylic acids and its salts and esters thereof;
polyacrylic acid/alkyl acrylate copolymers, preferably modified or unmodified carboxyvinyl polymers;
AMPS;
AMPS/acrylamide copolymers;
polyoxyethylenated AMPS/alkyl methacrylate copolymers;
anionic, cationic, amphoteric or nonionic chitin or chitosan polymers;
cellulose polymers and derivatives;
Starch polymers and derivatives, eventually modified;
vinyl polymers and derivatives;
polymers of natural origins and derivatives thereof;
alginates and carrageenans;
glycoaminoglycans, hyaluronic acid and derivatives thereof;
mucopolysaccharides such as hyaluronic acid and chondroitin sulfates;
and the mixtures thereof.

7. The composition of claim 1, wherein the microcapsules comprise at least:
a core made of at least one particle having a high wet point, the particle being optionally porous, and/or a monosaccharide-polyol, preferably mannitol,
at least two different layers,
at least one hydrophilic polymer preferably selected from polysaccharide or derivatives, and more preferably from starch or derivatives,
and advantageously at least one lipid based material, preferably an amphiphilic compound, more preferably a phospholipid, even more preferably phosphoacylglycerol such as hydrogenated lecithin.

8. The composition of claim 1, wherein the oil-absorbing filler is chosen from silicas, silica silylates, polyamide, acrylic polymer powders, especially polymethyl methacrylate, polymethyl methacrylate/ethylene glycol dimethacrylate, polyallyl methacrylate/ethylene glycol dimethacrylate or ethylene glycol dimethacrylate/lauryl methacrylate copolymer powders; perlites; magnesium carbonate, silicone filler and mixtures thereof.

9. The composition of claim 1, wherein the oil-absorbing filler is chosen from porous silica microspheres, polydimethylsiloxane-coated amorphous silica microspheres, silica silylate powders, amorphous hollow silica particles, precipitated silica powders surface-treated with a mineral wax, porous polymethyl methacrylate/ethylene glycol dimethacrylate spheres, ethylene glycol dimethacrylate/lauryl methacrylate copolymer powders, the hollow PMMA spheres, Nylon-6 powder, Nylon® 12, perlite powders, magnesium carbonate powders, organopolysiloxane powders, preferably coated with silicone resin; hollow hemispherical particles of silicone, hollow hemispherical particles of silicone.

10. A microcapsules composition comprising
releasable material
a core comprising at least one organic material,
at least one layered coating surrounding the core, the layered coating comprising a binder selected from at least one polymer, at least one lipid-based material, and their mixture, preferably their mixture and at least one of the particle(s), and
an outer layer comprising a hydrophilic polymer.

11. A method for preparing the microcapsule composition of claim 10 comprising;
preparing a solution containing a lower alcohol such as ethanol and a hydrophilic gelling agent which is soluble in water and the alcohol,
dispersing an aerogel and optionally a pigment in the aqueous solution; and
coating a core with the aqueous solution,
provided that the aqueous solution does not include any hydrophobic solvent.

12. The method of claim 11, wherein,
the hydrophilic gelling agent is hydroxypropylmethyl cellulose (HPMC).

13. The method of claim 11, wherein the solution contains water.
